(19) United States
(12) Patent Application Publication
(10) Pub. No.: US 2005/0038338 A1
(43) Pub. Date: Feb. 17, 2005

(54) Pre-operative planning of implantations

(76) Inventors: James Vincent Bono, Dover, MA (US); Anders Rosholm, Hellerup (DK); Ulrik Olesen Born, Vanlose (DK); Niels Baadegaard, Virum (DK); Rolf Sanne, Helsingborg (SE)

Correspondence Address: BACHMAN & LAPOINTE, P.C., 900 CHAPEL STREET, SUITE 1201, NEW HAVEN, CT, 06510, US

(21) Appl. No.: 10/847,560

(22) Filed: May 17, 2004

Related U.S. Application Data
(63) Continuation-in-part of application No. 10/722,526, filed on Nov. 28, 2003.
(60) Provisional application No. 60/494,254, filed on Aug. 11, 2003.

(51) Int. Cl. A61B 5/05

(52) U.S. Cl. 600/427

(57) ABSTRACT

A method and a system to be used pre-operatively to obtain guidance in the proper dimensions and model of implants to be implanted in a living body is described. The method includes the steps of determining a diameter of a template of a cup portion of a body part, determining a position of a center of said cup portion in an image of said body part, determining an orientation of said body part, and determining a location of a shaft of a bone associated with said body part. A system for performing the method is described.

### CROSS-REFERENCE TO RELATED APPLICATION(S)

The instant application claims the benefit of U.S. Provisional Patent Application Ser. No. 60/494,254, filed Aug. 11, 2003, entitled BIOMEDICAL DIGITAL TEMPLATING ALGORITHM. The present application is also a continuation-in-part application of pending U.S. patent application Ser. No. 10/722,526.

### BACKGROUND OF THE INVENTION

(1) Field of the Invention

The present invention relates to a method and a system to be used pre-operatively by surgeons to obtain guidance in the proper dimensions and model of implants to be surgically implanted in a living body.

(2) Prior Art

Presently, when preparing an implantation of a hip prosthesis, an appropriate model of the prosthesis and corresponding appropriate prosthesis dimensions are estimated, typically by the surgeon responsible for the operation. The goal is to find a prosthesis that optimally fits the patient prior to the operation.

Prosthesis models exist as 2-dimensional templates, depicting the contours of the prostheses as they appear in a relevant projection. The predetermination of an appropriate prosthesis reduces the risk for the surgeon of having to insert a number of different prostheses during the operation before finding one that actually fits the bone dimensions of the patient.

Due to their distance from the film media, bones and other objects are subject to varying degrees of magnification when imaged by X-ray equipment. For hip X-rays, large people with much soft tissue tends to have the imaged bones enlarged more than smaller people. This magnification effect implies that the object size observed in an X-ray typically differs from the real physical size. The difference in observed and real size should be taken in to account when finding appropriate prosthesis models based on the matching of two dimensional templates with the image contents.

In prior art systems, the determination of an appropriate template is often made based on a visual evaluation of a conventional X-ray of the relevant body part. Transparent two dimensional templates (printed actetates) of the possible prosthesis are manually overlaid by the surgeon onto the X-ray in order to visually evaluate whether the prosthesis will fit the body part to which it shall be attached or inserted in. The two dimensional templates depict the contours of the corresponding prosthesis calculated in the relevant projection direction of the X-ray.

When evaluating whether a template fits the bone part to which it shall be attached or inserted in, the transparent templates are manually moved and rotated by the user such that a subjectively best fit between the template and the relevant bone part is achieved.

Prior to the template evaluation, a certain magnification of the bones is assumed and the matched templates are chosen in a correspondingly enlarged scale. Typically, a magnification of 15 to 20% is assumed.

In addition to being relatively time consuming, the manual procedure for matching templates with the image contents have several drawbacks, including human operator imprecision and inter-operator variations.

The advent of digital (or digitized) X-rays has fundamentally changed the way that matching of templates may be carried out. The reading of X-rays is done from high-resolution computer screens and template matching using the transparent physical templates has effectively become impractical. Instead, the transparent physical templates have been substituted with digital versions of the templates that may be displayed as an overlay onto the screen displaying beneath the digital X-ray of the relevant body part.

In the digital set-up with both digital X-rays and digital templates, a manual procedure quite similar to the previous analog template matching procedure may be facilitated by enabling the operator (typically the surgeon) to load a digital template onto the screen and control its position and orientation using for example a computer mouse.

The correct scale of the digital templates is either estimated using the same ad-hoc assumptions as for conventional X-rays or through calibration of the digital image via the observed pixel size of an object in the image of known physical size. To achieve a correct magnification, estimated objects should be preferably placed in the same distance from the film media as the relevant bone.

The digital version of manually matching templates suffers from the same inconveniences as its analog counterpart, i.e. it is prone to imprecision and operator variability. The position and orientation of a digital template on the screen will further typically be controlled through mouse movements and mouse clicks, in which case the amount of time and mouse clicks used by the operator may be perceived as significantly inconvenient and inefficient. There is therefore a demand for tools, particularly software tools, that may assist the operator and reduce the time and effort to perform the template matching.

### SUMMARY OF THE INVENTION

Accordingly, it is an object of the present invention to provide an improved tool for assisting a surgeon to implant a prosthesis which is time efficient and requires reduced effort.

It is a further object of the present invention to provide a method and a system to be used pre-operatively by surgeons to obtain guidance in the proper dimensions and model of implants to be surgically implanted in a living body.

The foregoing objects are attained by the present invention.

In accordance with the present invention, a method for providing guidance on implants to be implanted into a living body broadly comprising the steps of determining a diameter of a template of a cup portion of a body part, determining a position of a center of said cup portion in an image of said body part, determining an orientation of said body part, and determining a location of a shaft of a bone associated with said body part.

Further, in accordance with the present invention, a system for providing guidance on implants to be implanted into a living body broadly comprises means for determining a diameter of a template of a cup portion of a body part, means for determining a position of a center of said cup portion displayed on an image of said body part, means for determining an orientation of said body part, and means for determining a location of a shaft of a bone associated with said body part.

Other details of the pre-operative planning of implantations, as well as other objects and advantages attendant thereto, are set forth in the following detailed description and the accompanying drawings wherein like reference numerals depict like elements.

### BRIEF DESCRIPTION OF THE DRAWINGS

FIG. 1 is an X-ray image of a hip joint illustrating the two first landmarks and a defined baseline;

FIG. 2 is an X-ray image illustrating the third and fourth landmarks indicating a desired cup center and cup diameter;

FIG. 3 is an X-ray image illustrating the fifth landmark which is indicative of a part of the femoral shaft where a stem prosthesis is approximately desired to be fitted into a medullar space;

FIG. 4 illustrates the initial region of interest (ROI) with the medullar (inner edges) detected;

FIG. 5 is representation of a refined shaft axis from an initial ROI being calculated and used as a middle column in a final shaft ROI;

FIG. 6 illustrates a final ROI with edges;

FIG. 7 illustrates the contours of a given digital template being matched with edges found within the final ROI;

FIG. 8 illustrates offset and leg-length discrepancy; and

FIG. 9 is a schematic representation of a hardware system in accordance with the present invention.

### DETAILED DESCRIPTION OF THE PREFERRED EMBODIMENT(S)

The present invention uses a computer and a dedicated software program that allows the user to mark certain landmarks on an image, generally an X-ray image, of a body part, such as a person's hip, which enables the system to define an appropriate region of interest (ROI) at the femoral shaft for which the implantation is relevant. Having defined an appropriate region of interest, the system then performs an edge detection analysis of the femoral shaft and matches this information with the contour information of a digital template. It further suggests an optimal position and orientation based on an appropriate match to the detected edges. Other similar embodiments of the present invention may be used to find prostheses for body parts other than hips, such as hand bones, knees, or other bones.

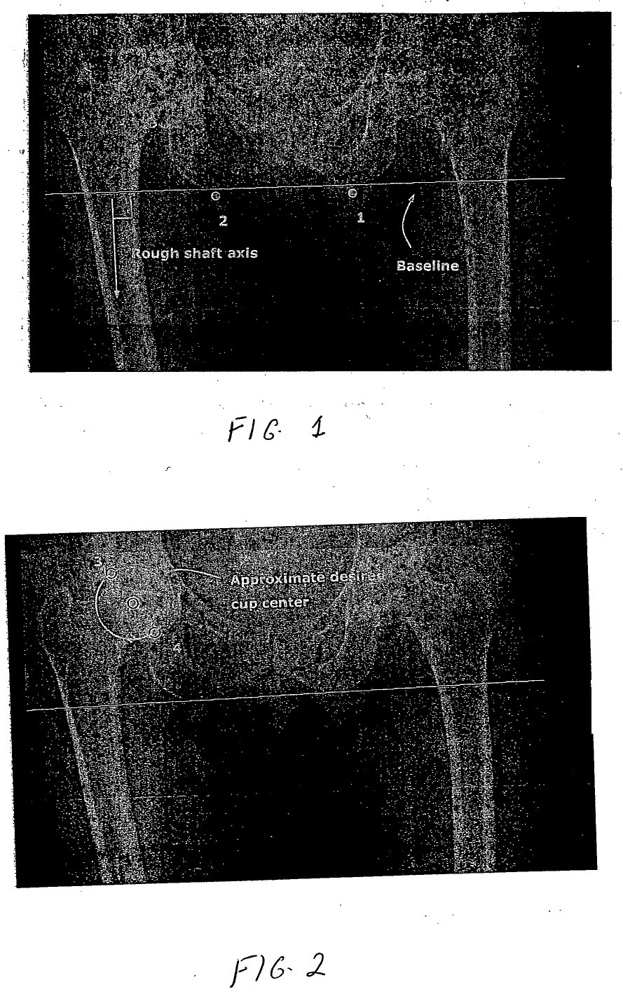

In the method of the present invention, the user initially marks two landmarks (reference numbers 1 and 2 in FIG. 1) at the pelvis using a pointing device, such as a mouse, to define a line denoted the baseline. The baseline is subsequently used by the system to initially make a rough estimate of the main direction or orientation of the femoral shaft. As shown in FIG. 1, the system displays the baseline.

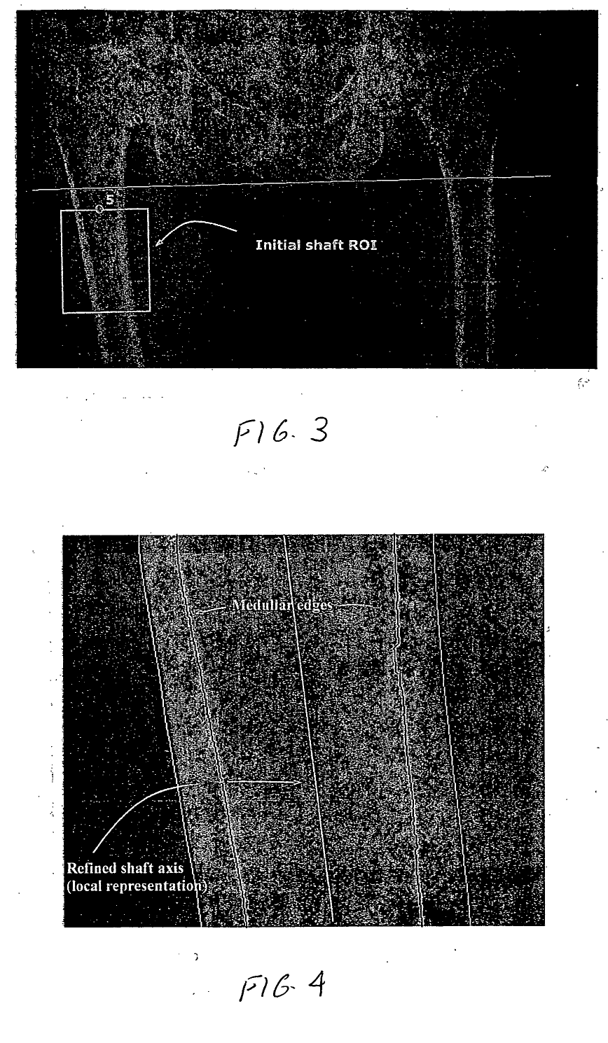

The user subsequently outlines, by two marked landmarks (reference numbers 3 and 4 in FIG. 2) on the acetabulum, an appropriate position and dimension of a cup prosthesis. From the two landmark points (3, 4), as shown in FIG. 2, the system defines a desired center of a cup template at the midpoint of the two points (3, 4) and with a cup diameter equal to the distance between the two points. A circle illustrating these uniquely defined cup properties are displayed. Again the landmark points are created through movement of a pointing device such as a mouse. The center of the cup is used later on to define positions of involved regions of interest.

The user then marks a point (reference number 5 in FIG. 3), using the pointing device, indicative of a part of the femoral shaft where a stem prosthesis approximately is desired to be fitted into the medullar space. The system extracts a rectangular region of interest (ROI) around the marked part of the femoral shaft. The direction orthogonal to the baseline is then used to define the main direction of the region of interest as shown in FIG. 3.

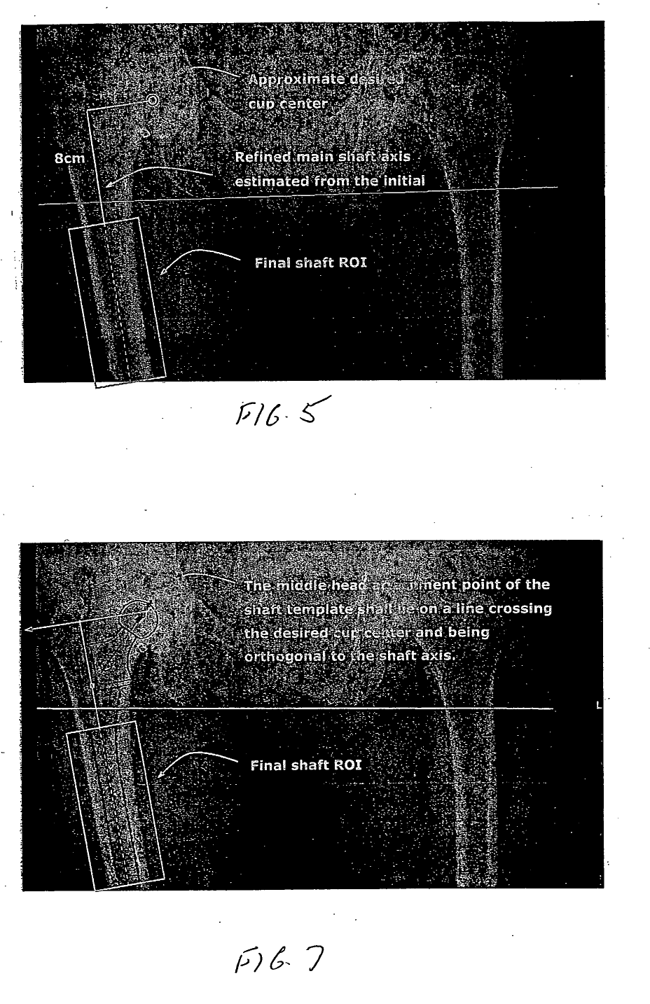

Referring now to FIG. 4, the femoral medullar (inner) edges of the region of interest are detected locally within the initial region of interest, associating a medullar edge point with the point of maximum intensity of the corresponding cortex. Based on the detected inner edges, a local refined shaft axis orientation is estimated (e.g. by fitting a line through the left and right edge points using orthogonal regression).

Referring now to FIG. 5, the global representation of the refined shaft axis from the initial region of interest is calculated and used as middle column in a final shaft region of interest. Thereby, the orientation of the final region of interest is defined. The top of the final shaft region of interest is positioned approximately 4.0 to 8.0 cm below the cup center defined in FIG. 2, calculated along the global shaft axis. The height of the final ROI is set so that the region of interest (ROI) extends to the image border as shown in FIG. 5.

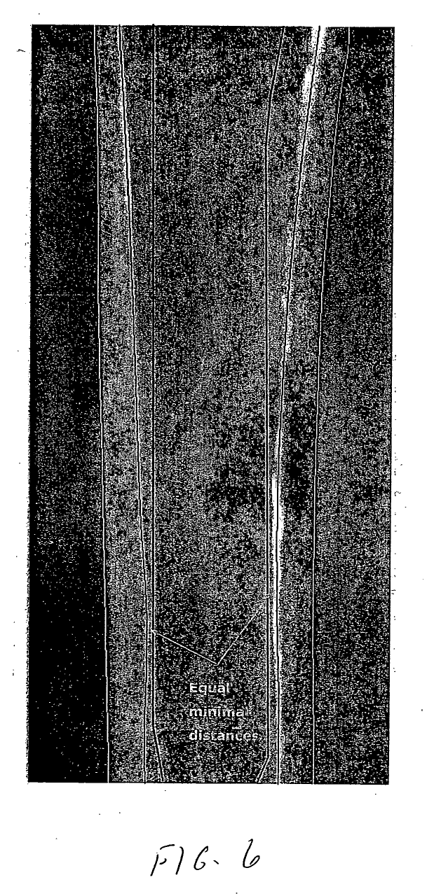

The femoral medullar (inner) edges of the region of interest are detected locally within the final region of interest, associating a medullar edge point with the point of maximum intensity of the corresponding cortex, as shown in FIG. 6. The contours of a digital template matched with the detected edges are shown. The template is positioned so that its horizontal position is optimized such that the contour of the digital template has the same distance to the left cortex edge as to the right cortex edge.

The contours of a given digital template are matched as shown in FIG. 7 with the edges found within the final region of interest. The main axis of the shaft template is kept parallel to the shaft axis, thereby determining the orientation of the template. As shown in FIG. 7, the position of the template along the shaft axis is fixed, such that the middle head attachment point of the stem template is on a line orthogonal to the shaft axis and going through the desired cup center. The remaining degree of freedom is the horizontal position of the template, which is optimized such that the contour of the digital template has the same minimal distance to the left cortex edge as to the right cortex edge (by horizontal is meant orthogonal to the shaft axis).

The fitness measure is calculated as the minimal horizontal distance between template contours and the cortex edges (by horizontal is meant orthogonal to the shaft axis).

After determining the optimal position of each template in a given set of available templates, the system suggests to the user the template with the best (lowest) fit.

The embodiment described above deals at least with positioning of a digital template of a prosthesis. The contours of the digital template is typically represented as x-and y-coordinates in a coordinate system with a pre-defined origo. The method is based on measurements of the width of the medullar space in a two dimensional projection image of the relevant bone into which the prosthesis is to be inserted, and the measurements are performed in a computerized manner in the sense that an image is digitized and loaded into a computer where one or more regions of interest are established typically by the before disclosed landmarking method.

Once a region of interest is defined/selected, the edges of the medullar space within the region of interest are detected, typically by applying an edge detecting algorithm which detects the edges preferably in an automated manner, that is without any interference or guidance from a user of the method. The medullar edges in a cross section of the bone is typically associated with the points of maximum intensity in the two dimensional projection image.

In the method, the main bone orientation is further estimated on the basis of the edges of the medullar space and/or the periosteal edges of the bone and the main bone orientation is in particular estimated by orthogonal regression through the medullar found edge points.

Following this step, a position of the template along, such as parallel to, the main bone orientation is determined. This is done in such a manner that a head attachment point of the shaft template lies on a line orthogonal to the bone axis is going through a desired cup prosthesis center.

Once the position along the bone is determined, an orientation of the digital template and a position orthogonal to the main bone orientation are determined. It is noted, that the step of find a position of the template along the main bone orientation and the step of determining the orientation of the digital template orthogonal to the main bone direction may be determined independently of each other resulting in that the steps do not have to be performed in a specific order. The orientation and position are determined such that the contours of the templates best possibly fit the detected edges of the medullar space. A fit may either be represented by the minimal or the average distance between the template contours and the found edges. Other definitions of a fit measure may further be applied. Also this step is preferably determined in an automated manner, and the fit between the template and the medullar edges is typically derived on the basis of minimizing the distances between the template contour and the edges.

The two dimensional projection image has preferably such a size that it covers at least part of the relevant bone and potentially extends to other body parts. As the method operates on a digital image, many types of images may be utilized including an X-ray image.

One aim of the method of the present invention is to suggest to a user, in an efficient manner requiring less user-interaction than with previous approaches, an appropriate position for a given template. However, the method may also advantageously be applied to select a template from a library of templates stored in a database The template may be a cup template stored in a library of cup templates or a stem template stored in a library of stem templates. In this regard, the method may be applied to a plurality of templates and a best fitting template, if present, is presented at its optimal position and rotation. A best fitting template is characterized as the one having the smallest distance between the contours and the edges. The templates are preferably stored in one or more databases from which they are loaded. After execution of the method on the plurality of templates, they are sorted according to the calculated fit for each template. Typically, a list of the sorted multiple templates is displayed to the user of the system.

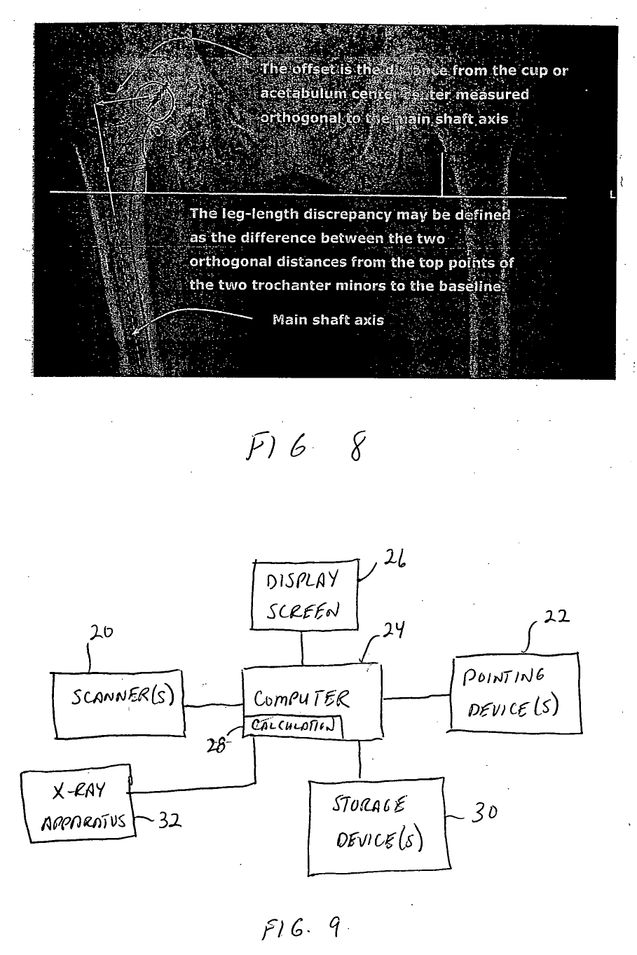

Prior to determining the template position and orientation, the method may assume a movement and/or orientation of the relevant bone as a result of the operation such that certain post-operational geometrical properties of the prosthesis is obtained. In particular, the relevant bone may be assumed moved and/or rotated during the operation such that a certain offset and/or leg length discrepancy are obtained as a result of the operation, an example of which is shown in FIG. 8 illustrating offset and leg-length discrepancy.

The user may specify that offset of the operated hip side should be the same as before the operation or the same as observed in the other side of the bone. The femoral shaft is then during the operation moved by the surgeon to a position that achieves this desired offset and the template position algorithm has to take this movement into account when matching the medullar edges of the shaft with the template contours. With respect to the leg length discrepancy, the user may likewise specify that it should be zero or at least be smaller than observed prior to the operation.

Referring now to FIG. 9, the method of the present invention makes use of a hardware system adapted to perform the steps of the method. This hardware system comprises a number of physical entities typically comprising one or more scanners 20 for digitizing images, one or more digital x-ray modalities, one or more pointing devices 22 operatively connected to a computer 24 with one or more screens 26 in such a manner that landmarks may be set and a visualization of the image with template as it appears from the accompanying figures. Further, the system comprises calculation devices 28 for performing numerical calculations and storage devices 30 storing the digital templates. Furthermore, the hardware system may advantageously make use of, comprise, or further comprise a digital X-ray apparatus 32 providing digitized X-ray images.

In operation, a user initiates the computer so that an image of the body part is displayed on a screen 26. The user then places an indicator over the left lower pelvis and right lower pelvis and clicks the pointing device 22 a first time when over the left lower pelvis and a second time when over the right lower pelvis to mark the landmarks 1 and 2 which mark-the baseline. The computer then displays the baseline on the screen 26. Thereafter, the user moves the indicator over the left lower pelvis and the right lower pelvis. The user clicks the pointing device a third time when over the upper periphery of the acetabulum and a fourth time when over the lower periphery of the acetabulum to mark the landmarks 3 and 4 which mark the edges of the acetabulum. Landmark 3 and 4 define in combination uniquely the desired center position of a cup as well as its preferred diameter. The desired cup center is defined to be precisely in between the landmarks and the preferred cup diameter is equal to the distance between the landmarks. A circle indicating these uniquely defined properties is displayed while the user marks the fourth landmark. The order of the 2 first landmarks may be interchanged.

The pre-programmed computer analyzes the landmarks 3 and 4 and displays a circle—representing the uniquely defined cup properties—on the screen 26 while the user marks the fourth position. When the positions are marked, the pre-programmed computer selects a cup template and inserts the cup template at the specified center position. If only a single cup template is present in a library, then it is selected as the default cup. If a plurality of cup templates of different sizes is present in the library, the one with a diameter closest to the specified diameter is selected. Instead of having the best cup template inserted automatically, the user may choose to be presented a list of the available cup templates (identified by their names) sorted according their fit. The user may then select a cup template from this list. This list may after the insertion of a first cup template be recalled on the screen so that the user can easily change templates.

The user then moves the pointing device 22 over the femoral shaft and clicks the pointing device 22 a fifth time to mark the femoral shaft. A stem template is selected and positioned in the area of the femoral shaft. The stem template may be selected by the pre-programmed computer from a default stem template or, if there are different sized stem templates, then the one that best fits the femoral canal is automatically inserted. Instead of having the best stem template inserted automatically, the user may choose to be presented a list of the available stem templates (identified by their names) sorted according their fit. The user may then select a stem template from this list. This list may after the insertion of a first stem template be recalled on the screen so that the user can easily change templates. Using the system of the present invention, due to the programming of the computer, the user can move, rotate, and change sizes and categories of templates as desired.

The cup and stem templates may be stored in a standard Windows file structure if desired or in a dedicated database structure. Administrating the template library may be done with a standard Windows Explorer or dedicated database administration tool.

The method and system of the present invention make it possible to do pre-operative planning and follow-ups after orthopedic surgery. The user can draw lines, measure distances, angles, etc., and get help determining which implant to use for surgery. The resulting image with the orthopedic template, annotations, and measurements can be saved for future comparison or printing.

It is apparent that there has been provided in accordance with the present invention pre-operative planning of implantations which fully satisfies the objects, means, and advantages set forth hereinbefore. While the present invention has been described in the context of specific embodiments thereof, other alternatives, modifications, and variations will become apparent to those skilled in the art having read the foregoing description. Accordingly, it is intended to embrace those alternatives, modifications, and variations as fall within the broad scope of the appended claims.

1. A method for providing guidance on implants to be implanted into a living body comprising the steps of:
determining an orientation of said body part;
determining a diameter of a template of a cup portion of a body part;
determining a position of a center of said cup portion on an image of said body part; and
determining a location of a shaft of a bone associated with said body part.

2. A method according to claim 1, wherein said body part includes the acetabulum and wherein said diameter determining step and said center position determining step comprises operating a pointing device so that an indicator is positioned over an upper periphery of the acetabulum; landmarking said upper periphery by operating said pointing device; operating said pointing device so that said indicator is positioned over a lower periphery of said acetabulum; and landmarking said lower periphery by operating said pointing device.

3. A method according to claim 1, wherein said orientation determining step comprises operating a pointing device to place an indicator over an image of a left lower pelvis, landmarking said left lower pelvis by operating said pointing device, operating said pointing device to move said indicator over an image of a right lower pelvis, and landmarking said right lower pelvis by operating said pointing device.

4. A method according to claim 1, wherein said shaft locating step comprises operating a pointing device to place an indicator over a femoral shaft and landmarking said femoral shaft by operating said pointing device.

5. A method according to claim 4, wherein said indicator placing step comprises placing said indicator on said image at a location at a distance in the range of from 4.0 cm. to 8.0 cm. below the trochanter minor.

6. A method according to claim 1, further comprising analyzing information about said cup diameter, said cup center position, said orientation of a hip, and said location of said femoral shaft and determining a cup template closest to said desired diameter.

7. A method according to claim 6, further comprising determining a stem template that fits within a medullar canal.

8. A method according to claim 6, wherein said cup template determining step comprises providing a database of cup templates and searching said database for a best fitting cup template.

9. A method according to claim 7, wherein said stem template determining step comprises providing a database of stem templates and searching said database for a best fitting stem template.

10. A method for assisting a surgeon performing a hip implant comprising the steps of:
electronically providing guidance in choice and placement of a cup template at an appropriate position and with an appropriate diameter relative to a size and position of an acetabulum; and
electronically providing guidance in choice and placement of a stem template at an appropriate position and with an appropriate stem width relative to the width and position of the femoral canal.

11. A method according to claim 10, wherein said cup template choice providing step comprises clicking said pointing device a first time on the left lower pelvis and clicking said pointing device a second time on the right lower pelvis to define a baseline indicative of an orientation of a hip in the image and wherein said stem template guidance providing step comprises clicking a pointing device a third time for defining an upper periphery of said acetabulum, clicking said pointing device a fourth time for defining a lower periphery of said acetabulum, and further clicking said pointing device a fifth time on a femoral shaft to determine where the femoral shaft is located.

12. A method according to claim 11, wherein said fifth clicking step comprises clicking the pointing device at a location from 4.0 cm to 8.0 cm below the trochanter minor.

13. A system for providing guidance on implants to be implanted into a living body broadly comprises:
means for determining an orientation of said body part;
means for determining a diameter of a template of a cup portion of a body part; and
means for determining a location of a shaft of a bone associated with said body part.

14. A system according to claim 13, wherein said body part includes the acetabulum and wherein said diameter determining means and said center position determining means comprises a pointing device which is operated so that an indicator is positioned over an upper periphery of the acetabulum and which is landmarked by operating said pointing device; and said pointing device being further operated so that said indicator is positioned over a lower periphery of said acetabulum and said lower periphery is landmarked by operating said pointing device.

15. A system according to claim 13, wherein said orientation determining means comprises a pointing device which is operated to place an indicator over an image of a left lower pelvis and which landmarks said left lower pelvis by operating said pointing device, and said pointing device further being operated to move said indicator over an image of a right lower pelvis, which is landmarked by operating said pointing device.

16. A system according to claim 13, wherein said shaft locating means comprises a pointing device which is operated to place an indicator over a femoral shaft and which landmarks said femoral shaft by operating said pointing device.

17. A system according to claim 16, wherein said indicator is placed on said image at a location at a distance in the range of from 4.0 cm. to 8.0 cm. below the trochanter minor.

18. A system according to claim 13, further comprising means for analyzing information about said cup diameter, said cup center position, said orientation of a hip, and said location of said femoral shaft and determining a cup template closest to said desired diameter.

19. A system according to claim 18, further comprising means for determining a stem template that fits within a medullar canal.

20. A system according to claim 18, wherein said cup template determining means includes a database of cup templates and means for searching said database for a best fitting cup template.

21. A system according to claim 19, wherein said stem template determining means includes a database of stem templates and means for searching said database for a best fitting stem template.
